(19) United States
(12) Plant Patent Application Publication
(10) Pub. No.: US 2002/0083498 P1
(43) Pub. Date: Jun. 27, 2002

(54) LILY PLANT NAMED 'TIBER'

(76) Inventor: CEES A. VAN DER VOORT, KATWIJK (NL)

Correspondence Address: FOLEY & LARDNER, WASHINGTON HARBOUR, 3000 K STREET, N.W., SUITE 500, WASHINGTON, DC, 20007-5109, US

(21) Appl. No.: 09/357,097

(22) Filed: Jul. 19, 1999

(51) Int. Cl. A01H 5/00

(52) U.S. Cl. PLT/315

(57) ABSTRACT

A new and distinct cultivar of Lily plant named ‘Tiber’ characterized by having upright to horizontal flowers, intense, pink color, contrasting white tepal ends, and vigorous growth habit.

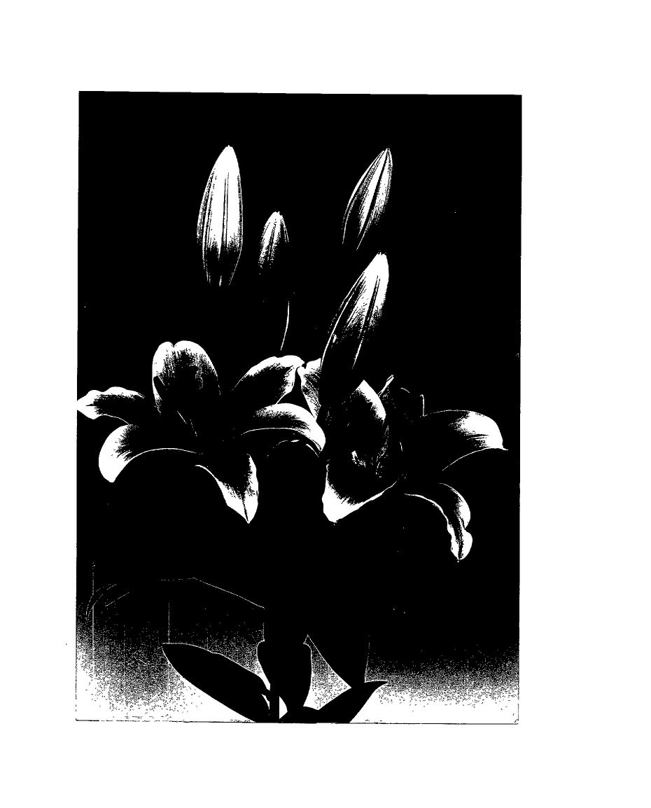

### BACKGROUND OF THE INVENTION

The present invention comprises a new and distinct cultivar of lily plant, botanically known as Lilium L., and hereinafter referred to by the cultivar name ‘Tiber’.

‘Tiber’ is a product of a planned breeding program which had the objective of creating new lily cultivars having large, upright flowers.

‘Tiber’ was originated from a hybridization made by the inventor in a controlled breeding program in Rijnsburg, the Netherlands in 1984. The male and female parents were unnamed seedlings of Lilium L. oriental hybrids. Both parents are proprietary cultivars used in the breeding program.

‘Tiber’ was discovered and selected as one flowering plant within the progeny of the stated cross by the inventor, Cees A. van der Voort, on Jun. 7, 1984 in a controlled environment in Rijnsburg, the Netherlands.

The first act of asexual reproduction of ‘Tiber’ was accomplished when scales were taken from the initial selection in October 1988 in a controlled environment in Rijnsburg, the Netherlands by, or under the supervision of, Cees A. van der Voort. Horticultural examination of selected units initiated in May, 1988 has demonstrated that the combination of characteristics as herein disclosed for ‘Tiber’ are firmly fixed and are retained through successive generations of asexual reproduction.

### BRIEF DESCRIPTION OF THE INVENTION

The following traits have been repeatedly observed and are determined to be basic characteristics of ‘Tiber’ which, in combination, distinguish this lily as a new and distinct cultivar:

1. upright to horizontal flowers;

2. intense, pink color;

3. contrasting white tepal ends; and

4. vigorous growth habit.

‘Tiber’ has not been observed under all possible environmental conditions. The phenotype of the new cultivar may vary significantly with variations in environment such as temperature, light intensity, and daylength without any change in genotype. The following observations, measurements, and comparisons describe plants grown at the experimental research institute CPRO-DLO, Wageningen, the Netherlands under conditions which approximate those generally used in commercial practice.

Of the many commercial cultivars known to the present inventor, no other cultivars are similar in comparison to ‘Tiber’.

### BRIEF DESCRIPTION OF THE DRAWING

The accompanying color photographic drawing shows typical plant, bulb and flower characteristics of ‘Tiber’, with colors being as true as possible with illustrations of this type.

### DETAILED BOTANICAL DESCRIPTION

In the following description, color references are made to The Royal Horticultural Society Colour Chart (RHS). The color values were determined between 12 am and 2 pm in May 1998 at the Dutch experimental research station CPRO-DLO, Wageningen, the Netherlands.

Origin: Seedling from breeding program.

Parentage:

Female parent.—Unnamed seedling.

Male parent.—Unnamed seedling.

Classification:

Botanical.—A hybrid of the genus Lilium L.

Commercial.—Oriental Lily cv. ‘Tiber’.

Plant:

Form.—A single stem carrying numerous, alternately arranged pedicels.

Height.—Medium to high for the mature plant, approximately 85 cm.

Growth Habit.—Vigorous.

Stem.—Circular in transverse section; anthocyanin pigmentation is present in spots and stripes; internodal length uniform.

Foliage:

Quantity.—Approximately 60 to 70 depending on cultivation conditions.

Size of leaf.—Mature leaves are approximately 7.5 cm long and 2.5 cm wide

Shape of leaf.—Generally ovate.

Color.—Medium green.

Bulb:

Size.—16 to 18 cm in circumference.

Color.—White/pink.

Bud:

Form.—Tapering like a lancet.

Size.—11 cm in length depending on cultivation conditions.

Opening.—Unfolds normally.

Color.—Light pink, RHS 62A

Tepals.—Folded, three visible.

Flower:

Blooming habit.—Raceme.

Size.—Medium, 20 cm in diameter depending on cultivation conditions

Borne.—Upright to horizontal along a longitudinal axis.

Shape.—Generally, hexagonal star in form and bowl-shaped in cross-section.

Tepalage:

Number of Tepals.—6.

Arrangement.—Three inner and three outer.

Color:

Upper Surface.—Pink, RHS 63C, mid-vein is green fading to yellow.

Lower Surface.—Light pink, RHS 62C.

Spotting or Marking.—Spots and markings are present, dark pink in color.

Pedicel:

Length.—Short to medium, 8 cm, depending on cultivation conditions.

Color.—Secondary color on tepal tips and edges is white, RHS 155D.

Form:

Texture.—Papillose.

Disease Resistance.—Medium.

Fragrance.—Weak.

Lasting Quality.—Excellent.

Reproductive organs:

Stamens and anthers:

Arrangements.—Anthers form a generally circular pattern at their upper ends.

Number.—6.

Length.—7 cm depending on cultivation conditions.

Filaments Color.—Yellowish green.

Pollen color.—Dark brown.

Anther color.—Dark brown.

Pistils.—Green.

Stigma.—Grey.

Ovaries.—Triangular in shape.

I claim:

1. A new and distinct cultivar of Lily plant named ‘Tiber’, as illustrated and described herein.
